(19) United States
(12) Patent Application Publication
(10) Pub. No.: US 2017/0000891 A1
(43) Pub. Date: Jan. 5, 2017

(54) FOOD COMPOSITION

(71) Applicant: FUJIFILM CORPORATION, Tokyo (JP)

(72) Inventor: Masao SATO, Kanagawa (JP)

(21) Appl. No.: 15/172,159

(22) Filed: Jun. 3, 2016

(30) Foreign Application Priority Data
Jun. 30, 2015 (JP) 2015-131090

(51) Int. Cl. A61K 47/26; A61K 31/122; A61K 31/202; A61K 9/48; A23D 9/007; A61K 47/34

(52) U.S. Cl. CPC A61K 47/26; A23L 1/293; A23L 1/3002; A23L 1/302; A23L 1/304; A23L 1/3016; A23L 1/0014; A23D 9/007; A61K 47/34; A61K 31/202; A61K 9/48; A61K 31/122; A23V 2002/00

(57) ABSTRACT

A food composition comprising astaxanthin (A), a powder (B) of a sucrose fatty acid ester that is powdery at 25° C. and has an HLB value of 10 or more, and an oil (C) that is a liquid at 25° C., wherein the powder (B) is dispersed in a dispersion medium comprising the oil (C).

### CROSS-REFERENCE TO RELATED APPLICATIONS

This application is based on and claims priority under 35 USC 119 from Japanese Patent Application No. 2015-131090, filed on Jun. 30, 2015. The entire disclosure of the above application is incorporated herein by reference.

### BACKGROUND

Technical Field

The present invention relates to a food composition.

Related Art

Astaxanthin has attracted attention as a component having ‘anti-oxidant action’ for eliminating active oxygen. Astaxanthin is known to have various functions such as immunostimulation, fatigue recovery and balance adjustment of an autonomic nerve, and is widely distributed also as a raw material for health foods.

However, astaxanthin is insoluble in water and has the defect of bad internal absorbency. Thus, in recent years, a technique for improving absorbability of astaxanthin in the body has been proposed.

For example, in Japanese Patent Application Laid-Open (JP-A) Nos. 2009-62330 and 2009-62331, a technique for powdering an emulsion, which is obtained by heating and mixing an oily phase component containing astaxanthin and an aqueous phase component containing sucrose fatty acid ester, lecithin and the like, by spray drying is disclosed.

### SUMMARY

The absorbability of astaxanthin in the body can be improved by a technique for spray drying, which is disclosed in JP-A Nos. 2009-62330 and 2009-62331. On the other hand, it is also desired that a technique in the place of spray drying allows a food composition such that the absorbability of astaxanthin in the body is equivalent or higher to conventional products.

The present invention has been made in view of the circumstances as described above, and the problem to be solved of some embodiments of the invention is to provide a food composition excellent in the absorbability of astaxanthin in the body.

A specific method for solving the problem includes the following aspects.

<1>A food composition including astaxanthin (A), a powder (B) of a sucrose fatty acid ester that is powdery at 25° C. and an HLB value of 10 or more, and an oil (C) that is a liquid at 25° C., wherein the powder (B) is dispersed in a dispersion medium including the oil (C).

<2>The food composition according to <1>, further including a polyoxyethylene sorbitan fatty ester (D) with an HLB value of 10 or more.

<3>The food composition according to <2>, wherein the polyoxyethylene sorbitan fatty ester (D) is polyoxyethylene sorbitan monolaurate.

<4>The food composition according to any one of <1>to <3>, wherein the powder (B) is a powder of sucrose laurate.

<5>The food composition according to any one of <1>to <4>, further including crocetin.

<6>The food composition according to any one of <1>to <5>, wherein a dosage form of the food composition is a soft capsule.

Some embodiments of the invention can provide a food composition excellent in the absorbability of astaxanthin in the body.

### BRIEF DESCRIPTION OF THE DRAWINGS

FIG. 1 is a graph showing a result of an absorption test of astaxanthin performed for a food composition of Examples 1 to 2 and Comparative Example 1.

### DETAILED DESCRIPTION

An example of the embodiments of a food composition to which the present invention is applied is hereinafter described. However, the invention is not limited to the following embodiments at all, and may be performed under proper modifications within the scope of the object of the invention.

In the specification, the range of a numerical value denoted by using ‘to’ signifies a range including the numerical values described before and after ‘to’ as the minimum value and the maximum value respectively.

In the specification, the amount of each component in the food composition signifies a total amount of plural kinds of substances existing in the food composition unless otherwise specified in the case where a substance corresponding to each component exists by plural kinds in the food composition.

In the specification, ‘HLB (Hydrophile-Lipophile Balance) value’ adopts a value described in a document such as a catalogue in the case where a commercial product is used as the component and an HLB value of the commercial product is definitely described in a document such as a catalogue of the commercial product.

In the case where the component to be used is not a commercial product, or in the case where an HLB value is not definitely described in a document such as a catalogue even though the component is commercially available, an HLB value in the specification adopts a value obtained by the calculation formula of Griffin. In the calculation formula of Griffin, an HLB value is calculated in accordance with the following formula by using a value of S (saponification value of ester) and a value of N (neutralization value of fatty acid composing ester). The HLB value signifies more hydrophilic property as the value is closer to 20, and signifies more lipophilic property as the value is closer to 0.

HLB value=20 (1−S/N)

In the specification, the expression ‘glycerin fatty ester’ includes a glycerin fatty ester containing one glycerin unit and one fatty acid unit, a glycerin fatty ester containing a plurality of a glycerin unit and one fatty acid unit or a plurality of a fatty acid unit and one glycerin unit, and a glycerin fatty ester containing a plurality of a glycerin unit and a plurality of a fatty acid unit, and the expression is used in the case of using these glycerin fatty esters without any distinction.

In the specification, the term ‘step’ includes not merely an independent step but also a step whose expected object is achieved even in the case of not being capable of distinguishing definitely from other steps.

[Food Composition]

A food composition of the embodiment is a food composition containing astaxanthin (A), powder (B) of a sucrose fatty acid ester that is powdery at 25° C. and having an HLB value of 10 or more (also referred to as ‘powder (B) of a sucrose fatty acid ester’ hereinafter) and an oil (C) that is a liquid at 25° C. (also referred to as ‘an oil (C)’ hereinafter), in which the powder (B) of the sucrose fatty acid ester is dispersed in a dispersion medium containing the oil (C) that is a liquid at 25° C.

The food composition of the embodiment is excellent in the absorbability of astaxanthin in the body.

The reason why the food composition of the embodiment is excellent in the absorbability of astaxanthin in the body is not clear but the inventors of the invention presume as follows.

Astaxanthin is insoluble in water and has poor absorbability in the body. On the other hand, it is conceived that a sucrose fatty acid ester with an HLB value of 10 or more has an affinity for water in the body and has good absorbability.

In the food composition of the embodiment, it is conceived that the coexistence of astaxanthin and a sucrose fatty acid ester with an HLB value of 10 or more in a dispersion medium containing an oil that is a liquid at 25° C. causes sucrose fatty acid ester to complement the affinity of astaxanthin for water and causes the absorbability of astaxanthin in the body to become excellent.

The components contained in the food composition of the embodiment are hereinafter described in detail.

<Astaxanthin (A)>

The food composition of the embodiment contains astaxanthin (A).

In the food composition of the embodiment, the astaxanthin (A) includes at least one selected from astaxanthin or derivatives thereof (such as ester of astaxanthin). In the specification, astaxanthin and derivatives thereof are generically named ‘astaxanthin’.

Synthetic products of astaxanthin obtained in accordance with an ordinary method in addition to astaxanthin derived from natural products such as plants, algae, crustaceans and bacteria may be also used as the astaxanthin (A).

The astaxanthin may be extracted from cultures such as red ferment Phaffia, green algae haematococcus, marine bacteria and krill.

From the viewpoint of quality and productivity, the astaxanthin (A) is preferably astaxanthin derived from an extract from haematococcus algae (hereinafter referred to as ‘haematococcus algae extract’) or an extract from krill, particularly preferably astaxanthin derived from haematococcus algae extract.

In the food composition of the embodiment, the astaxanthin (A) is preferably in a state of being dissolved in an oil.

Specific examples of the haematococcus algae include Haematococcus pluvialis, Haematococcus lacustris, Haematococcus capensis, Haematococcus droebakensis and Haematococcus zimbabwiensis.

Among these, the haematococcus algae are preferably Haematococcus pluvialis.

The haematococcus algae extract may be obtained in such a manner that the cell wall of the haematococcus algae is crushed as required by the method disclosed in JP-A No. 5-68585 to add organic solvents such as acetone, ether, chloroform and alcohols (for example, ethanol and methanol), or extraction solvents such as carbon dioxide in a super critical state.

A commercial product may be used as the haematococcus algae extract.

Examples of the commercial product of the haematococcus algae extract include ASTOTS (registered trademark)-S, ASTOTS (registered trademark)-5 O and ASTOTS (registered trademark)-10 O of FUJIFILM Healthcare Laboratory Co., Ltd., ASTAREAL (registered trademark) OIL50F and ASTAREAL (registered trademark) OIL5F of Fuji Chemical Industries Co., Ltd., and BIOASTIN SCE7 of Toyokosokagaku Inc.

The content of astaxanthin as true content of coloring matter in the haematococcus algae extract is preferably 0.001% by mass to 50% by mass, more preferably 0.01% by mass to 25% by mass from the viewpoint of handling during production.

Incidentally, the haematococcus algae extract may contain astaxanthin or an ester form thereof as true content of coloring matter similarly to the coloring matter described in JP-A No. H2-49091.

The content of the astaxanthin (A) is preferably 0.001% by mass or more, more preferably 0.005% by mass or more with respect to the whole amount of the food composition from the viewpoint of sufficiently obtaining an effect expected by containing the astaxanthin (A), for example.

Also, the content of the astaxanthin (A) is preferably 10% by mass or less, more preferably 5% by mass or less with respect to the whole amount of the food composition from the viewpoint of ingestibility, for example.

<Powder (B) of Sucrose Fatty Acid Ester>

The food composition of the embodiment contains a powder (B) of a sucrose fatty acid ester that is powdery at 25° C. and has an HLB value of 10 or more. The inclusion of the powder (B) of the sucrose fatty acid ester in the food composition of the embodiment improves the absorbability of astaxanthin in the body.

The food composition of the embodiment may contain the powder (B) of the sucrose fatty acid ester singly by one kind or by combination of two kinds or more.

In the food composition of the embodiment, the powder (B) of sucrose fatty acid ester is dispersed in a dispersion medium containing an oil (C) that is a liquid at 25° C. The sucrose fatty acid ester is powdery at 25° C. The sucrose fatty acid ester is favorably dispersed in the dispersion medium containing an oil (C) due to being powdery at 25° C.

The powder (B) of a sucrose fatty acid ester is preferably such a size as to pass through a filter with an opening of 180 μm (approximately 83 meshes), more preferably such a size as to pass through a filter with an opening of 150 μm (approximately 100 meshes).

The HLB value of the sucrose fatty acid ester is 10 or more, preferably 12 or more, more preferably 14 or more from the viewpoint of the absorbability of astaxanthin in the body.

Also, the HLB value of the sucrose fatty acid ester is preferably 19 or less from the viewpoint of exhibiting amphipathic property.

The carbon number of a fatty acid composing the sucrose fatty acid ester is preferably 8 or more, more preferably 10 to 18 from the viewpoint of exhibiting amphipathic property, for example.

Specific examples of the sucrose fatty acid ester include sucrose laurate, sucrose myristate, sucrose palmitate, sucrose stearate and sucrose oleate.

Among these, the sucrose fatty acid ester is preferably at least one kind selected from sucrose laurate, sucrose palmitate or sucrose stearate, particularly preferably sucrose laurate from the viewpoint of the absorbability of astaxanthin in the body.

Examples of a commercial product of the powder (B) of sucrose fatty acid ester include RYOTO (registered trademark) SUGAR ESTER S1170 (trade name, sucrose stearate, HLB value: approximately 11 (catalogue value), Mitsubishi-Kagaku Foods Corporation), RYOTO (registered trademark) SUGAR ESTER S1570 (trade name, sucrose stearate, HLB value: approximately 15 (catalogue value), Mitsubishi-Kagaku Foods Corporation), RYOTO (registered trademark) SUGAR ESTER S1670 (trade name, sucrose stearate, HLB value: approximately 16 (catalogue value), Mitsubishi-Kagaku Foods Corporation), RYOTO (registered trademark) SUGAR ESTER P1570 (trade name, sucrose palmitate, HLB value: approximately 15 (catalogue value), Mitsubishi-Kagaku Foods Corporation), RYOTO (registered trademark) SUGAR ESTER P1670 (trade name, sucrose palmitate, HLB value: approximately 16 (catalogue value), Mitsubishi-Kagaku Foods Corporation), and RYOTO (registered trademark) SUGAR ESTER L1695 (trade name, sucrose laurate, HLB value: approximately 16 (catalogue value)).

The content of the powder (B) of sucrose fatty acid ester is preferably 0.1% by mass or more, more preferably 0.5% by mass or more with respect to the whole amount of the food composition from the viewpoint of, for example, the absorbability of astaxanthin in the body.

Also, the content of the powder (B) of the sucrose fatty acid ester is preferably 30% by mass or less, more preferably 20% by mass or less with respect to the whole amount of the food composition from the viewpoint of ingestibility, for example.

The content of the powder (B) of the sucrose fatty acid ester is preferably 100 parts by mass or more, more preferably 300 parts by mass or more with respect to 100 parts by mass of the astaxanthin (A) from the viewpoint of, for example, the absorbability of astaxanthin in the body.

Also, the content of the powder (B) of the sucrose fatty acid ester is preferably 10000 parts by mass or less, more preferably 8000 parts by mass or less with respect to 100 parts by mass of the astaxanthin (A) from the viewpoint of ingestibility, for example.

The sucrose fatty acid ester having an HLB value of 10 or more exists in a powdery state in the dispersion medium containing an oil (C) due to not being dissolved in oil. Accordingly, if the presence of the powder of the sucrose fatty acid ester may be confirmed in the dispersion medium containing an oil (C), the HLB value of the sucrose fatty acid ester is conceived to be 10 or more. Examples of a confirmation method include an optical microscope, centrifugal separation, solvent (such as a solvent for dissolving an oily component without dissolving the sucrose fatty acid ester) extraction, and combination thereof.

Also, the sucrose fatty acid ester having an HLB value of 10 or more is not dissolved in oil, so that it may be confirmed by analyzing a water extract of an object while using a liquid chromatographic method (LC: Liquid Chromatography), a mass spectrometric method (MS: Mass Spectrometry), an infrared spectroscopic method (IR: Infrared Spectroscopy), a nuclear magnetic resonance method (NMR: Nuclear Magnetic Resonance), and combination thereof that the sucrose fatty acid ester is contained in the object.

<Oil (C)>

The food composition of the embodiment contains an oil (C) that is a liquid at 25° C. In the food composition, the oil (C) functions as a dispersion medium for a powder (B) of sucrose fatty acid ester.

The food composition may contain the oil (C) singly by one kind or by combination of two kinds or more.

In the food composition, the oil (C) is not particularly limited if the oil is generally used for food and is liquid at 25° C.

In the specification, ‘an oil that is a liquid at 25° C.’ signifies an oil having a melting point of less than 25° C.

Examples of the oil (C) include oils and fats, and higher fatty acids.

Examples of the oils and fats include oils and fats derived from plants, such as olive oil, avocado oil, corn oil, rapeseed oil, yolk oil, sesame oil, wheat germ oil, linseed oil, safflower oil, cottonseed oil, soybean oil, peanut oil, teaseed oil, kaya oil, rice bran oil, germ oil, safflower oil (namely, Carthamus tinctorius oil), palm oil, coconut oil, almond oil, sasanqua oil, camellia oil, evening primrose oil and grapefruit seed oil. Also, examples of the oils and fats of (C) include tocopherol and tocotrienol which are extracted from these oils and fats derived from plants.

The higher fatty acids are not particularly limited if they are fatty acids with a carbon number of 8 or more, and examples thereof include lauric acid, myristic acid, palmitic acid, stearic acid, oleic acid, behenic acid, linoleic acid, linolenic acid, eicosapentaenoic acid and docosahexaenoic acid.

Among these, the oil (C) is preferably at least one kind selected from safflower oil or coconut oil in the view points of being capable of stably compounding each component (such as astaxanthin and a powder (B) of sucrose fatty acid ester) contained in the food composition, and allowing liquid physical properties (such as viscosity) suitable for soft capsulation in the case of selecting a soft capsule as a dosage form of the food composition. The oil (C) is particularly preferably safflower oil in view of industrial handleability.

The content of the oil (C) is preferably 100 parts by mass or more, more preferably 200 parts by mass or more with respect to 100 parts by mass of the astaxanthin (A) from the viewpoint of handling during production, for example.

Also, the content of the oil (C) is preferably 10000 parts by mass or less, more preferably 8000 parts by mass or less with respect to 100 parts by mass of the astaxanthin (A) from the viewpoint of ingestibility, for example.

<Polyoxyethylene Sorbitan Fatty Ester (D)>

The food composition of the embodiment preferably further contains a polyoxyethylene sorbitan fatty ester (D) having an HLB value of 10 or more. The food composition of the embodiment is further improved in terms of the absorbability of astaxanthin in the body by additional inclusion of the polyoxyethylene sorbitan fatty ester (D).

In the case of containing the polyoxyethylene sorbitan fatty ester (D), the food composition of the embodiment may contain the polyoxyethylene sorbitan fatty ester (D) singly by one kind or by combination of two kinds or more.

The HLB value of the polyoxyethylene sorbitan fatty ester is preferably 10 or more, more preferably 12 or more.

Also, the HLB value of the polyoxyethylene sorbitan fatty ester is preferably 19 or less from the viewpoint of exhibiting amphipathic property.

The average addition number of oxyethylene groups of the polyoxyethylene sorbitan fatty ester is preferably 10 to 30, preferably 15 to 25 from the viewpoint of hydrophilic and hydrophobic property balance, for example.

Examples of the polyoxyethylene sorbitan fatty ester include polyoxyethylene sorbitan monolaurate, polyoxyethylene sorbitan monopalmitate, polyoxyethylene sorbitan monostearate and polyoxyethylene sorbitan monooleate.

Among these, the polyoxyethylene sorbitan fatty ester is preferably polyoxyethylene sorbitan monolaurate from the viewpoint of the absorbability of astaxanthin in the body.

Examples of a commercial product of the polyoxyethylene sorbitan fatty ester (D) include WILSURF TF-20 (trade name, polyoxyethylene sorbitan monolaurate, HLB value: 16.7 (catalogue value), NOF CORPORATION.), WILSURF TF-60 (trade name, polyoxyethylene sorbitan monostearate, HLB value: 15.7 (catalogue value), NOF CORPORATION.), and WILSURF TF-80 (trade name, polyoxyethylene sorbitan monooleate, HLB value: 15.7 (catalogue value), NOF CORPORATION.).

The content of the polyoxyethylene sorbitan fatty ester (D) is preferably 20 parts by mass or more, more preferably 50 parts by mass or more with respect to 100 parts by mass of the astaxanthin from the viewpoint of the absorbability of astaxanthin in the body, for example.

Also, the content of the polyoxyethylene sorbitan fatty ester (D) is preferably 10000 parts by mass or less, more preferably 8000 parts by mass or less with respect to the whole amount of the food composition from the viewpoint of ingestion dose to easy consume, for example.

<Other Components>

The food composition of the embodiment may contain other components other than astaxanthin (A), a powder (B) of a sucrose fatty acid ester and an oil (C) as essential components, and a polyoxyethylene sorbitan fatty ester (D) as an optional component within a range of not spoiling the effect of the invention as required.

Examples of the other components include components allowed to be generally used for food.

Examples of such components include an emulsifying agent (excluding the powder (B) of the sucrose fatty acid ester and the polyoxyethylene sorbitan fatty ester (D) described above), an antifoaming agent, an antioxidant, a stabilizer, a sweetener, an acidulant, a preservative, a colorant and a perfume.

Also, examples of the other components include functional components whose ingestion is expected to bring beneficial effects to health (effects such as health maintenance, health promotion, and prevention and improvement of lifestyle-related illnesses).

Examples of such functional components include various kinds of vitamins, minerals, crocetin, collagen (such as hydrolytic collagen and water-soluble collagen), ornithine, resveratrol, chlorogenic acid, caffeic acid, ubiquinone (such as coenzyme Q10), flavonoid (such as flavanone, flavone, flavonol, isoflavone, catechin and anthocyanin), lignan (such as sesamin and sesamolin) and curcumine. The functional components may be plant-derived components, animal-derived components, fermentation products by yeast, or chemical synthetic products suitable for food.

(Crocetin)

The food composition of the embodiment preferably further contains crocetin.

Crocetin is one kind of carotenoid and may be obtained by hydrolyzing crocin contained in the fruit of a gardenia and the stigma of a saffron.

The further inclusion of crocetin in the food composition of the embodiment allows additional effects, such as eyestrain improvement effect and sleep improvement effect, to be expected.

In the case where the food composition of the embodiment contains crocetin, the content of crocetin is not particularly limited and may be determined properly in accordance with kinds and degrees of the expected effects.

(Emulsifying Agent)

The food composition of the embodiment may contain an emulsifying agent. The inclusion of an emulsifying agent in the food composition allows the powder (B) of a sucrose fatty acid ester to be contained more stably in a dispersion medium containing an oil (C).

In the case of containing an emulsifying agent, the food composition of the embodiment may contain an emulsifying agent singly by one kind or by combination of two kinds or more.

The emulsifying agent is not particularly limited and is preferably a nonionic detergent, for example.

Examples of the nonionic detergent include glycerin fatty ester and polyglycerin condensed ricinoleate.

Among these, the nonionic detergent is preferably a glycerin fatty ester with an HLB value of 15 or less, more preferably a glycerin fatty ester with an HLB value of 1 to 10 from the viewpoint of exhibiting amphipathic property, for example.

The glycerin fatty ester is preferably an ester of polyglycerin having an average degree of polymerization of 1 to 10 and fatty acid having a carbon number of 8 to 18, such as caprylic acid, capric acid, lauric acid, myristic acid, palmitic acid, stearic acid, oleic acid or linoleic acid.

Examples of a commercial product of the glycerin fatty ester include EMAX (registered trademark) BW-36 (trade name, HLB value: 3 to 5, RIKEN VITAMIN CO., LTD.), SY GLYSTER (registered trademark) MS-3 S (trade name, tetraglycerin monostearate, HLB value: 8.4, Sakamoto Yakuhin kogyo Co., Ltd.), SY GLYSTER (registered trademark) MO-3S (trade name, tetraglycerin monooleate, HLB value: 8.8, Sakamoto Yakuhin kogyo Co., Ltd.), and SY GLYSTER (registered trademark) PO-5S (trade name, hexaglycerin pentaoleate, HLB value: 4.7, Sakamoto Yakuhin kogyo Co., Ltd.).

In the case where the food composition of the embodiment contains an emulsifying agent, the content of the emulsifying agent is preferably 1 part by mass to 1000 parts by mass with respect to 100 parts by mass of the oil (C) from the viewpoint of dispersion stability of the powder (B) of the sucrose fatty acid ester in a dispersion medium containing the oil (C), for example.

<Dosage Form>

Examples of a dosage form of the food composition of the embodiment include a soft capsule, a cream and a mousse.

Among these, the dosage form of the food composition of the embodiment is preferably a soft capsule. The soft capsule as the dosage form of the food composition allows the dispersion of the powder (B) of the sucrose fatty acid ester to be maintained. Also, the stability of each component contained in the food composition can be retained. In addition, smell derived from components contained in the food composition, such as astaxanthin, can be shielded.

<Application>

The food composition of the embodiment is so excellent in the absorbability of astaxanthin in the body having various functions such as immunomodulation, fatigue recovery and balance adjustment of an autonomic nerve as to be capable of being appropriately applied as health foods, functional foods and dietary supplements.

[Producing Method for Food Composition]

A producing method for the food composition of the embodiment is not particularly limited and a publicly known method may be used. The food composition of the embodiment may be produced, for example, by the method described below.

The food composition may be produced by a method including the mixing (also referred to as ‘mixing step’ hereinafter) of astaxanthin (A), a powder (B) of a sucrose fatty acid ester and an oil (C), and other components (including the optional component described above) as required.

A mixing method of astaxanthin (A), the powder (B) of the sucrose fatty acid ester and the oil (C) is not particularly limited; for example, all components may be mixed at one time. Astaxanthin (A) and the oil (C) may be mixed to obtain an oil solution, and thereafter the obtained oil solution and the powder (B) of the sucrose fatty acid ester may be mixed. The powder (B) of the sucrose fatty acid ester and the oil (C) may be mixed to obtain an oil solution, and thereafter the obtained oil solution and astaxanthin (A) may be mixed.

It is preferable from the viewpoint of favorably dispersing the powder (B) of a sucrose fatty acid ester in a dispersion medium containing an oil (C) that astaxanthin (A) and the oil (C) are mixed to obtain an oil solution, and thereafter the obtained oil solution and the powder (B) of a sucrose fatty acid ester are mixed.

The mixing method is not particularly limited. Any commercial mixing equipment may be used. Examples of the mixing equipment include mixing equipment such as a stirrer and a mixer.

The conditions such as temperature and time in mixing are not particularly limited but may be adjusted properly in accordance with kinds of components contained in the food composition. For example, the temperature in mixing while containing the powder (B) of a sucrose fatty acid ester is preferably determined at a temperature less than the melting point of the sucrose fatty acid ester due to needing to be a temperature at which the sucrose fatty acid ester may retain the form of powder.

The producing method for the food composition may include an other step other than the mixing step as required. Examples of the other step include a comminuting step of comminuting the powder (B) of a sucrose fatty acid ester as a pretreatment step before the mixing step.

Examples of equipment for the comminuting treatment include equipment suitable for dry comminution of food ingredients, such as a pulverizer, a grinder, a crusher and a mill.

The comminuting treatment of the powder (B) of a sucrose fatty acid ester is performed at preferably from 5° C. to 25° C., more preferably from 10° C. to 20° C. in a state of maintaining the temperature of the powder (B) of a sucrose fatty acid ester, from the viewpoint of restraining stickiness.

The comminuting treatment of the powder (B) of a sucrose fatty acid ester is performed so that 90% or more of comminuted products pass through preferably a filter with an opening of 180 μm (approximately 83 meshes), more preferably a filter with an opening of 150 μm (approximately 100 meshes).

The producing method for the food composition may include formulation steps and the like, such as a step of sealing the mixed liquid (also referred to as ‘content liquid’ hereinafter) obtained in the mixing step into a capsule membrane and a step of drying the capsule membrane into which the content liquid is sealed, in the case of making the dosage form of the food composition into a soft capsule.

The capsule membrane preferably contains gelatin, glycerin and water.

The content of glycerin in the capsule membrane is, for example, preferably 10 parts by mass to 70 parts by mass with respect to 100 parts by mass of gelatin.

Examples of a method for sealing the content liquid into the capsule membrane include publicly known methods such as a rotary type (for example, a rotary die type), a seamless type and a flat board type.

A method for drying the capsule membrane into which the content liquid is sealed is not particularly limited and a publicly known dryer such as a tumbler dryer (that is, a rotating-drum dryer) may be used.

The conditions such as temperature and time in drying are not particularly limited but may be adjusted properly in accordance with kinds of components contained in the food composition and components contained in the capsule membrane.

The soft capsule is preferably further dried after drying by a dryer. In this case, the drying temperature is preferably approximately from 20° C. to 30° C., the humidity is preferably approximately 10% RH to 50% RH, and the drying time is preferably approximately three days to ten days.

The form of the soft capsule is not particularly limited but any form such as OVAL, OBLONG and ROUND may be taken. Known methods or equipment in the industry may be applied for taking these forms.

### EXAMPLES

The invention is hereinafter described more specifically by examples, and is not limited to the following examples unless it deviates from the gist thereof.

[Production of Food Composition]

### Example 1

34.5 parts by mass of ASTOTS-100 (trade name, haematococcus algae-derived astaxanthin oil, astaxanthin concentration: 10% by mass, FUJIFILM Healthcare Laboratory Co., Ltd.) and 92 parts by mass of safflower oil (edible oils and fats, Nisshin OilliO Group, Ltd.) were sufficiently mixed at 45° C. by using a stirrer to obtain an oil solution of astaxanthin.

Next, RYOTO (registered trademark) SUGAR ESTER L1695 (trade name, sucrose laurate, HLB value: approximately 16, Mitsubishi-Kagaku Foods Corporation) was subject to comminuting treatment by using a dry pulverizer while maintaining a temperature of from 10° C. to 20° C. to obtain a comminuted product of RYOTO (registered trademark) SUGAR ESTER L1695 (also referred to as ‘a comminuted product of sucrose fatty acid ester’ hereinafter). It was confirmed that 90% or more of the obtained comminuted products of the sucrose fatty acid ester passed through a filter with an opening of 150 μm (approximately 100 meshes).

25 parts by mass of the obtained comminuted products of the sucrose fatty acid ester and 25 parts by mass of safflower oil (edible oils and fats, Nisshin OilliO Group, Ltd.) were sufficiently mixed at room temperature (25° C.) by using a stirrer, and the obtained mixture and the whole amount of the oil solution of astaxanthin obtained in the above were sufficiently mixed at room temperature (25° C.) to obtain a food composition of Example 1.

### Example 2

34.5 parts by mass of ASTOTS-100 (trade name, haematococcus algae-derived astaxanthin oil, astaxanthin concentration: 10% by mass, FUJIFILM Healthcare Laboratory Co., Ltd.), 89 parts by mass of safflower oil (edible oils and fats, Nisshin OilliO Group, Ltd.), and 3 parts by mass of WILSURF TF-20 (trade name, polyoxyethylene sorbitan monolaurate, HLB value: 16.7 (catalogue value), NOF CORPORATION.) were sufficiently mixed at 45° C. by using a stirrer to obtain an oil solution of astaxanthin.

Next, 25 parts by mass of the comminuted products of the sucrose fatty acid ester obtained in the same manner as Example 1 and 25 parts by mass of safflower oil (edible oils and fats, Nisshin OilliO Group, Ltd.) were sufficiently mixed at room temperature (25° C.) by using a stirrer, and the obtained mixture and the whole amount of the oil solution of astaxanthin obtained in the above were sufficiently mixed at room temperature (25° C.) to obtain a food composition of Example 2.

### Comparative Example 1

34.5 parts by mass of ASTOTS-100 (trade name, haematococcus algae-derived astaxanthin oil, astaxanthin concentration: 10% by mass, FUJIFILM Healthcare Laboratory Co., Ltd.) and 142 parts by mass of safflower oil (edible oils and fats, Nisshin OilliO Group, Ltd.) were sufficiently mixed at 45° C. by using a stirrer to obtain an oil solution of astaxanthin.

The obtained oil solution of astaxanthin was regarded as a food composition of Comparative Example 1.

[Evaluations]

<Absorbability of Astaxanthin>

The absorbability of astaxanthin was evaluated for each food composition of Examples 1 and 2 and Comparative Example 1 by the following method.

Each food composition of Examples 1 and 2 and Comparative Example 1 was orally dosed to a male eight-week-old rat which has been subject to one-week acclimatization. The oral dosing adopted single forced dosing by using a stomach sonde of a general flexible type. The dose of the food composition was determined so that the astaxanthin amount per 1 kg of the weight of the rat was 20 mg. The number of the rats in each test group was determined at four. With regard to all individuals, 1 mL of blood was taken from a vein under no anesthesia at each time of 0.5 hour, 1 hour, 2 hours, 3 hours, 4 hours, 6 hours, 8 hours and 24 hours after the dosing. Plasma was separated from the taken blood, and then the astaxanthin amount which was extracted from the separated plasma was analyzed by using high-performance liquid chromatography (HPLC) manufactured by SHIMADZU CORPORATION, and quantified by an absolute calibration curve method. The HPLC analysis conditions are shown below.

˜HPLC Analysis Conditions˜

Column: CAPSULE PACK C8 (inside diameter: 4.6 mm×length: 150 mm, Shiseido Japan Co., Ltd.)

Mobile phase: A liquid) methanol:water=75:25, B liquid) methanol=100

Gradient cycle: A liquid+B liquid=100%. The composition of B liquid is denoted below.

B liquid=20%→100% (0 minute→15 minutes)

B liquid=100%→100% (15 minutes→20 minutes)

B liquid=100%→20% (20 minutes→21 minutes)

B liquid=20%→20% (21 minutes→30 minutes)

Flow rate: 1 mL/minute

Column temperature: 40° C.

Injection amount: 1 μL

Detection wavelength: 470 nm

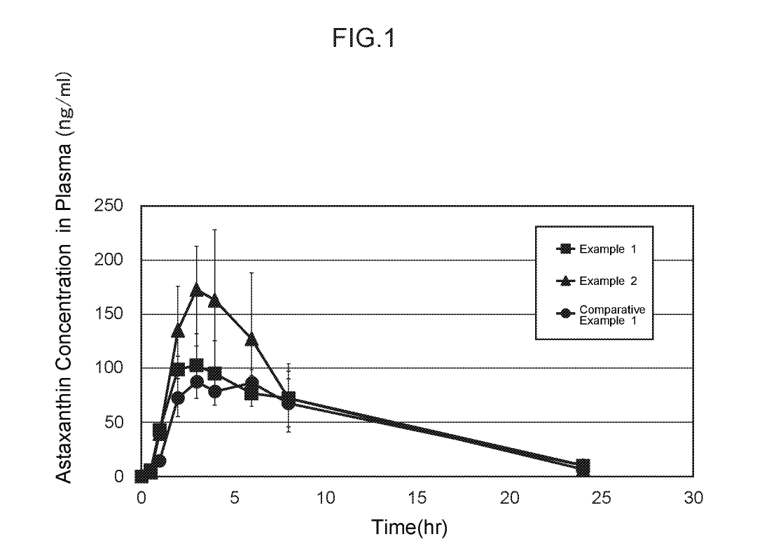

The change of astaxanthin concentration in the blood plasma is shown in FIG. 1. In FIG. 1, the vertical axis denotes astaxanthin concentration in the blood plasma (unit: ng/mL) and the horizontal axis denotes time after the dosing (unit: hr).

AUC (area under the curve) from 0 hour to 24 hours after the dosing is shown in Table 1. Incidentally, the numerical value of AUC adopted a value such that the measurement results of the four rats were averaged to calculate to the first decimal place by rounding off.

The larger numerical value of AUC signifies more excellent absorbability of astaxanthin in the body.

TABLE 1

| | AUC |
|---|---|
| EXAMPLE 1 | 1247.1 |
| EXAMPLE 2 | 1541.1 |
| COMPARATIVE EXAMPLE 1 | 1150.6 |

As shown in FIG. 1, in the food composition of Examples 1 and 2, the concentration of astaxanthin in the plasma rose in a short time as compared with the food composition of Comparative Example 1. This rise in the concentration of astaxanthin in the plasma in a short time was observed more remarkably in the food composition of Example 2.

Also, as shown in Table 1, in the food composition of Examples 1 and 2, the numerical value of AUC was large as compared with the food composition of Comparative Example 1, and such a tendency was observed more remarkably in the food composition of Example 2.

From the above results, it was clarified that the coexistence of astaxanthin and a powder of a sucrose fatty acid ester that is powdery at 25° C. and has an HLB value of 10 or more in a dispersion medium containing an oil that is a liquid at 25° C. improved the absorbability of astaxanthin in the body.

Also, it was clarified that the use of polyoxyethylene sorbitan fatty ester having an HLB value of 10 or more together with a powder of a sucrose fatty acid ester having an HLB value of 10 or more improved the absorbability of astaxanthin more remarkably.

[Formulating of Food Composition: Soft Capsule Formulation]

### Formulation Example 1

116.19 parts by mass of safflower oil (edible oils and fats, Nisshin OilliO Group, Ltd.) and 15 parts by mass of EMAX (registered trademark) BW-36 (trade name, glycerin fatty ester, HLB value: 3 to 5, RIKEN VITAMIN CO., LTD.) were sufficiently mixed by using a stirrer while heated at 60° C. to obtain a uniform oil solution. It was visually confirmed that the oil solution was uniform.

This oil solution was cooled to 50° C., and thereafter 7 parts by mass of SY GLYSTER CRS-75 (trade name, polyglycerin condensated ricinoleate, Sakamoto Yakuhin kogyo Co., Ltd.) and 2.25 parts by mass of E-MIX-α 1000 (trade name, vitamin E (tocopherol), TAMA BIOCHEMICAL CO., LTD.) were added thereto and mixed, and 34.5 parts by mass of ASTOTS-100 (trade name, haematococcus algae-derived astaxanthin oil, astaxanthin concentration: 10% by mass, FUJIFILM Healthcare Laboratory Co., Ltd.), 4 parts by mass of SY GLYSTER MO-3 S (trade name, tetraglycerin monooleate, Sakamoto Yakuhin kogyo Co., Ltd.), and 3 parts by mass of WILSURF TF-20 (trade name, polyoxyethylene sorbitan monolaurate, HLB value: 16.7 (catalogue value), NOF CORPORATION.) were further added and sufficiently mixed to obtain a mixture. A stirrer was used for mixing.

6.25 parts by mass of LALMIN Se2000 (trade name, mineral yeast, mineral: Se (selenium), MIWA SEIYAKU CO., LTD.), 25 parts by mass of beer yeast containing zinc by 10% (trade name, beer yeast, containing zinc by 10% by mass, Kelatron Corporation), 6.25 parts by mass of R-α lipoic acid CD (trade name, cyclodextrin clathrates of a lipoic acid R body, Cyclochem Co., Ltd.), 25 parts by mass of vitamin C (trade name, FUSO CHEMICAL CO., LTD.), and 5.56 parts by mass of sesame extract SESAVITA (registered trademark) (trade name, White Sesamin Extract 90% by mass, Bio Actives Japan Corporation) were added to the obtained mixture and sufficiently mixed by using a stirrer to thereafter comminute the obtained mixture by using a wet mill and obtain a comminuted product.

Next, RYOTO (registered trademark) SUGAR ESTER L1695 (trade name, sucrose laurate, HLB value: approximately 16, Mitsubishi-Kagaku Foods Corporation) was subject to dry comminuting treatment by using a comminutor while maintaining a temperature of from 10° C. to 20° C. to obtain a comminuted product of RYOTO (registered trademark) SUGAR ESTER L1695 (a comminuted product of sucrose fatty acid ester). It was confirmed that 90% or more of the obtained comminuted products of the sucrose fatty acid ester passed through a filter with an opening of 150 μm (approximately 100 meshes).

25 parts by mass of the obtained comminuted products of the sucrose fatty acid ester and 25 parts by mass of safflower oil (edible oils and fats, Nisshin OilliO Group, Ltd.) were mixed at room temperature (25° C.) by using a mixer, and the obtained mixture and the whole amount of the comminuted product obtained in the above were sufficiently mixed by using a stirrer at room temperature (25° C.), and the obtained mixture was used as a content liquid of a soft capsule.

Next, while feeding a gelatin sheet containing 35 parts by mass of glycerin with respect to 100 parts by mass of gelatin to a die roll, the content liquid was dropped by 300 mg in a sheet junction to produce an OVAL-shaped soft capsule. The produced soft capsule was subjected to tumbler drying and ventilation drying to obtain a soft capsule formulation. The soft capsule formulation after drying weighed 460±30 mg per one capsule.

The defects such as flaws and cracks were not observed in the obtained soft capsule formulation.

### Formulation Example 2

A soft capsule formulation was produced in the similar manner as Formulation Example 1 except for modifying the used amount of safflower oil (edible oils and fats) from 141.19 parts by mass to 136.69 parts by mass and the used amount of WILSURF TF-20 (polyoxyethylene sorbitan monolaurate) from 3 parts by mass to 7.5 parts by mass.

### Formulation Example 3

A soft capsule formulation was produced in the similar manner as Formulation Example 1 except for modifying the used amount of safflower oil (edible oils and fats) from 141.19 parts by mass to 144.19 parts by mass and not using WILSURF TF-20 (polyoxyethylene sorbitan monolaurate).

### Formulation Example 4

A soft capsule formulation was produced in the similar manner as Formulation Example 1 except for modifying the used amount of safflower oil (edible oils and fats) from 141.19 parts by mass to 114.95 parts by mass, the used amount of EMAX (registered trademark) BW-36 (glycerin fatty ester) from 15 parts by mass to 10.5 parts by mass, the used amount of beer yeast containing zinc by 10% (beer yeast) from 25 parts by mass to 50 parts by mass, and further using 5.74 parts by mass of CRAVIT P (trade name, crocetin, RIKEN VITAMIN CO., LTD.) together with LALMIN Se2000 (mineral yeast) and the like.

1. A food composition comprising astaxanthin (A), a powder (B) of a sucrose fatty acid ester that is powdery at 25° C. and has an HLB value of 10 or more, and an oil (C) that is a liquid at 25° C.,
wherein the powder (B) is dispersed in a dispersion medium comprising the oil (C).

2. The food composition according to claim 1, further comprising a polyoxyethylene sorbitan fatty ester (D) having an HLB value of 10 or more.

3. The food composition according to claim 2, wherein the polyoxyethylene sorbitan fatty ester (D) is polyoxyethylene sorbitan monolaurate.

4. The food composition according to claim 1, wherein the powder (B) is a powder of sucrose laurate.

5. The food composition according to claim 2, wherein the powder (B) is a powder of sucrose laurate.

6. The food composition according to claim 3, wherein the powder (B) is a powder of sucrose laurate.

7. The food composition according to claim 1, further comprising crocetin.

8. The food composition according to claim 2, further comprising crocetin.

9. The food composition according to claim 3, further comprising crocetin.

10. The food composition according to claim 4, further comprising crocetin.

11. The food composition according to claim 1, wherein a dosage form of the food composition is a soft capsule.
